(19) United States
(12) Patent Application Publication
(10) Pub. No.: US 2026/0165628 A1
(43) Pub. Date: Jun. 18, 2026

(54) DEVICE AND METHOD FOR DETERMINING ELECTROPHYSIOLOGICAL RESPONSE

(71) Applicant: Aycoutay Technologies Inc., Halifax (CA)

(72) Inventor: Dimitri POLITYKO, Lakehurst (CA)

(21) Appl. No.: 19/126,693

(22) PCT Filed: Nov. 2, 2023
(86) PCT No.: PCT/CA2023/051466

§ 371 Date: May 2, 2025

Related U.S. Application Data
(60) Provisional application No. 63/421,696, filed on Nov. 2, 2022.

(51) Int. Cl. A61B 5/256; A61B 5/00; A61N 1/36

(52) U.S. Cl. CPC A61B 5/256; A61B 5/6823; A61B 5/6831; A61B 2562/043; A61N 1/36034

(57) ABSTRACT

Device and method for measuring electrophysiological responses of a subject. The device comprises at least one front sensor to be positioned at (z.e., in electrical contact with) a subject's torso and at least one rear sensor to be positioned at the subject's back. In some embodiments, the device comprises a plurality of front sensors arranged vertically down the subject's torso and a corresponding plurality of rear sensors arranged vertically down the subject's back. A stimulus pulse is delivered through one of the sensors (e. g, a front sensor) from a control unit on the device. Responses to the stimulus pulse are measured at other sensors (e.g, sensors at the subject's back). The gathered response data is analyzed. In some embodiments, the device comprises a harness that sits over at least one of the subject's shoulders.

### TECHNICAL FIELD

The present invention relates to electrophysiology. More specifically, the present invention relates to stimulating and measuring electrophysiological responses in animals, including humans.

### BACKGROUND

As is well-known, the electrophysiological response of animal tissue measures electrical properties, or action potential activity, within biological cells and tissues, and the effects of that electrical activity on the body. At the cellular level, that electrical activity (i.e., electrophysiological activity) consists of the movement of charges (ions) through cell surface membranes at the synaptic junction between cells. Abnormalities in cell membrane integrity, resulting from injury, infection, disease, and/or changes or deficiencies in nutritional status, can modify the function of ion channels. Modified ion channel functions results in irregular electrical activity, as the movement of the ions through the cell membranes is altered and/or impaired. In turn, the irregular electrical activity results in irregular cellular communication and, hence, irregular physiological function throughout the affected tissue.

Electrophysiological tests are used to analyze such electrical activity to assist with diagnosing such abnormalities, injuries, etc. For example, ECG procedures (“electrocardiogram” procedures) are used to measure electrical responses within cardiac tissue. Numerous other organ-specific testing procedures are known in the art and include, without limitation, electrooculography (EOG, for measuring responses of the entire eye), electromyography (EMG, for measuring muscular responses), electrodermatology (EDG, for measuring responses of the skin), electrohepatology (EHG, for measuring responses of the liver), and many others.

As well as organ-specific responses, electrophysiological responses that indicate communication among organs and systems have been investigated in humans for more than two decades (see reference [1], detailed below, which describes the functional and chemical anatomy of the afferent vagal system).

Electrophysiological data related to such multi-organ responses, and particularly changes in trends of such data, may be useful in diagnostics, wellness monitoring, the personalization of preventive medicine, and the monitoring of bodily responses to individual's environments.

However, currently, it is difficult to monitor such responses. Existing tools are generally complex and inconvenient to use. For example, devices where sensors are to be applied ‘wet’ (i.e., where gels are used to improve sensor conductivity) are difficult and messy for home use. Additionally, where sensors are positioned manually, it can be difficult for subjects to ensure placement consistency between different scans. Similarly, systems that require supervision and/or interpretation by health care providers are not convenient for home tracking of potential trends by subjects themselves.

As such, there is a need for devices and methods that are convenient for subjects to gather electrophysiological data relating to multi-organ systems.

### SUMMARY

This document discloses a device for measuring electrophysiological responses of a subject, and a method of using such a device. The device comprises at least one front sensor to be positioned at (i.e., in electrical contact with) a subject's torso and at least one rear sensor to be positioned at the subject's back. In some embodiments, the device comprises a plurality of front sensors arranged vertically down the subject's torso and a corresponding plurality of rear sensors arranged vertically down the subject's back. A stimulus pulse is delivered through one of the sensors (e.g., a front sensor) from a control unit on the device. Responses to the stimulus pulse are measured at other sensors (e.g., sensors at the subject's back). The gathered response data is analyzed. In some embodiments, the device comprises a harness that sits over at least one of the subject's shoulders.

In a first aspect, this document discloses a device for measuring an electrophysiological response of a subject to predetermined electrical stimulus, said device comprising: a front extension; a rear extension; at least one connecting member, said at least one connecting member connecting said front extension and said rear extension; at least one front sensor attached to said front extension; and at least one rear sensor attached to said rear extension, wherein, when said device is in use: said front extension extends vertically down a front of said subject's torso; said rear extension extends vertically down said subject's back; and said at least one front sensor and said at least one rear sensor are in electrical contact with said subject's skin, and wherein a processor coupled with the device determines said electrophysiological response based on data from said at least one front sensor and said at least one rear sensor.

In another embodiment, this document discloses a device further comprising a control unit that: directs the delivery of power to at least one of said at least one front sensor and said at least one rear sensor; and gathers data from at least one of said at least one front sensor and said at least one rear sensor.

In another embodiment, this document discloses a device wherein a number of sensors on said front extension and a number of sensors on said rear extension are equal.

In another embodiment, this document discloses a device wherein said at least one front sensor and said at least one rear sensor each comprises a plurality of sensors.

In another embodiment, this document discloses a device wherein said at least one front sensor and said at least one rear sensor are positioned at predetermined locations on said subject's torso.

In another embodiment, this document discloses a device wherein said at least one connecting member comprises a strap.

In another embodiment, this document discloses a device wherein at least one of said front extension and said rear extension is conformable to said subject's body.

In another embodiment, this document discloses a device wherein said device further comprises a chest piece, said front extension extending from said chest piece and said chest piece being connected to said rear extension by said at least one connecting member.

In another embodiment, this document discloses a device wherein said device further comprises a back piece, said rear extension extending from said back piece and said back piece being connected to said front extension by said at least one connecting member.

In another embodiment, this document discloses a device wherein said device further comprises a harness, said harness having a chest piece and a back piece, said chest piece and said back piece being connected by said at least one connecting member, said front extension extending from said chest piece and said rear extension extending from said back piece, and said harness being positioned over at least one shoulder of said subject when said device is in use.

In another embodiment, this document discloses a device having a harness wherein said harness has a unitary construction.

In another embodiment, this document discloses a device wherein said plurality of sensors are is activated in a predetermined activation sequence.

In another embodiment, this document discloses a device wherein said sequence comprises: an activation of a sensor on said front extension to thereby provide a first stimulus pulse, and a determination of a first response, said first response being a response to said first stimulus pulse, from each said at least one rear sensor, wherein activations and response-determinations are performed for each other sensor on said front extension such that responses from each said at least one rear sensor are determined for each at least one front sensor.

In another embodiment, this document discloses a device wherein said sequence comprises: an activation of a sensor on said rear extension to thereby provide a first stimulus pulse, and a determination of a first response, said first response being a response to said first stimulus pulse, from each said at least one front sensor, wherein activations and response-determinations are performed for each other sensor on said rear extension such that responses from each said at least one front sensor are determined for each at least one rear sensor.

In another embodiment, this document discloses a device further comprising at least one additional sensor, wherein said at least one additional sensor is connected to a control unit on said device and wherein said control unit is in communication with said processor.

In a second aspect, this document discloses a method for measuring an electrophysiological response of a subject to predetermined electrical stimulus, said method comprising: activating a sensor positioned at a torso of said subject to thereby provide a first stimulus pulse; and determining a response to said first stimulus pulse from at least one sensor positioned at a back of said subject.

In another embodiment, this document discloses a method wherein a plurality of front sensors are positioned at said subject's torso and wherein steps (a) and (b) are separately repeated for each of said plurality of front sensors.

In another embodiment, this document discloses a method wherein each of said plurality of front sensors is vertically aligned with other sensors of said plurality of front sensors, and wherein said plurality of front sensors extends down said subject's torso.

In another embodiment, this document discloses a method wherein a uppermost sensor of said plurality of front sensors is activated first and each next-highest sensor of said plurality of front sensors is activated in sequence.

In another embodiment, this document discloses a method wherein a number of said at least one sensor positioned at said subject's back is equal to a number of said plurality of front sensors.

In a third aspect, this document discloses a method for measuring an electrophysiological response of a subject to predetermined electrical stimulus, said method comprising: activating a sensor positioned at a back of said subject to thereby provide a first stimulus pulse; and determining a response to said first stimulus pulse from at least one sensor positioned at a torso of said subject.

### BRIEF DESCRIPTION OF THE DRAWINGS

The present invention will now be described by reference to the following figures, in which identical reference numerals refer to identical elements and in which:

FIG. 1 is a schematic of a device according to one aspect of the invention;

FIG. 2A is a front view of a device according to another embodiment of the invention;

FIG. 2B is a rear view of the device of FIG. 2A;

FIG. 2C is a perspective view of the device of FIG. 2A;

FIGS. 3A and 3B show the device of FIG. 2A in use by a subject; and

FIG. 4 is a flowchart detailing a method according to another aspect of the invention.

### DETAILED DESCRIPTION

To better understand the present invention, the reader is directed to the listing of citations at the end of this description. For ease of reference, these citations and references have been referred to by their listing number throughout this document. The contents of the citations in the list at the end of this description are hereby incorporated by reference herein in their entirety.

The present invention provides a device for stimulating and determining an electrophysiological response of human tissues. The response data can be used for diagnostics, general wellness monitoring, etc., and relates to multi-organ interactions. That is, the electrophysiological responses measured do not need to be focused on any single organ.

The device comprises at least one sensor for positioning at a front of a subject's torso and at least one sensor for positioning at the subject's back. As should be understood, the term “sensor” as used herein refers to any device, piece of equipment, or component that can deliver and/or sense a current. This includes without limitation wet or dry electrodes, wet or dry pads, wet or dry terminals, etc. The sensors/electrodes/pads/terminals are, in some embodiments, suitable for gathering electrical current information from the surface of the subject's skin. In other embodiments, the sensors are alternatively or additionally suitable for gathering subcutaneous electrical current information. Similarly, the sensors/electrodes/pads/terminals are, in some embodiments, suitable for delivering electrical stimuli (such as electrical pulses/electrical currents) to the subject's skin. As well, as should be clear, the phrase “positioned at [location]”, as used herein, indicates that the sensor is in effective electrical contact with the subject's skin at the location. Effective electrical contact, moreover, means that pulses can be delivered and response data gathered as described (e.g., from the skin surface and/or subcutaneously).

As should be clear, the subject may be a human subject or another animal subject. The specific configuration and implementation of the device, of course, may vary depending on the nature of the subject. Animal subjects may include, without limitation, household and/or farm animals; industrial animals; laboratory animals; monitored wildlife animals; vertebrates and invertebrates; etc. As further non-limiting examples, subjects could include cows, dogs, cats, horses, mice, rats, birds, etc.

In one embodiment, at least one of the sensor(s) on the subject's front is used to deliver a stimulus pulse. Responses to the stimulus pulse(s) are measured from each sensor on the subject's back. As would be understood, as the path between a single front sensor and two separate rear sensors would be different, responses may differ between different rear sensors for a single stimulus pulse. The specific positions for each sensor depend on the desired measurements to be taken. In some embodiments, each front sensor is used to deliver stimulus pulses to the subject's torso. In one embodiment, further, pulses are delivered from sensors on the subject's front in descending order (i.e., the first pulse is delivered by the uppermost sensor, the next pulse by the next-lowest sensor, and so on) and responses to the pulses are recorded at each sensor on the subject's back.

However, as should be understood, depending on the embodiment, pulses can be delivered in any desired order. Additionally, in some embodiments, pulse(s) are delivered by sensors at the subject's back and response(s) measured by sensors at the subject's front. In still further embodiments, pulses can be delivered and/or responses measured at both the subject's back and front. In still further embodiments, all front and back sensors are used for gathering response data and the pulse(s) are delivered by a sensor that is at another location on the subject's body (i.e., neither the front nor the back of the torso).

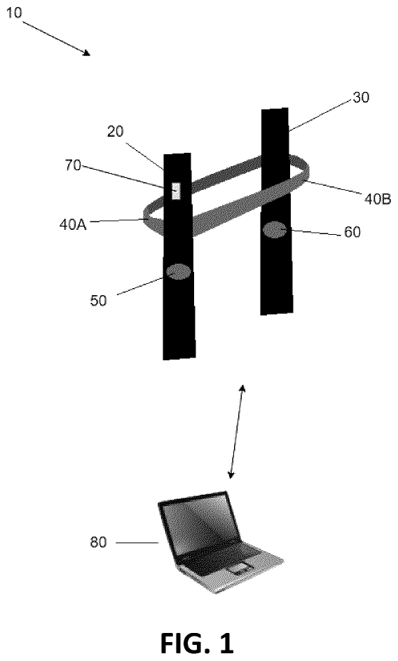

FIG. 1 shows a schematic of one embodiment of the invention. Device 10 comprises a front extension 20 and a rear extension 30. The front extension 20 and rear extension 30 are connected to each other by at least one connecting member 40A, 40B. A front sensor 50 is attached to the front extension 20 and a rear sensor 60 is attached to the rear extension 30. A communications and control unit 70, which is electrically connected to the front sensor 50 and rear sensor 60 by wires (not shown), directs the delivery of stimulus pulses and the gathering of response data. The control unit 70 is also in communication with a processor 80 for analyzing the gathered response data and determining the response.

In use, the front extension 20 extends vertically down the subject's torso. In one embodiment, the front extension 20 is centrally aligned with the subject's torso (i.e., bisecting the torso and generally aligned with the subject's sternum), while in other embodiments, the front extension 20 is offset from the centre of the torso. The subject can determine a suitable positioning for the front extension 20. Similarly, in use, the rear extension 30 extends vertically down the subject's back and, in one embodiment, bisects the subject's torso at the rear (i.e., is typically aligned with the subject's spine). In other embodiments, the rear extension 30 is offset from the centre of the torso.

As well, in some embodiments, the front extension 20, the rear extension 30, or both are at least partially pliable, so that they can be shaped and conformed to different subjects'bodies. In some such embodiments, the entire front extension 20 and/or rear extension 30 are plastic and conformable to subject's bodies, i.e., made out of a plastic material. In other such embodiments, the front extension 20 and/or rear extension 30 comprise pliable/deformable portions and non-pliable/non-deformable portions.

The length of the front extension 20 and rear extension 30 generally depends on the desired number and placement of sensors. For example, if one front sensor is to be positioned at the subject's sternum and another front sensor is to be positioned below the subject's navel, the front extension 20 must be long enough for such positioning to be feasible. As such, in some embodiments, the front extension 20 and/or rear extension 30 are extendable, e.g., by telescoping, deforming, or adding additional sections.

In some embodiments, the front extension 20 and/or the rear extension 30 have specific attachment points for sensor(s). Such embodiments may be preferred over embodiments without such attachment points, so that the positions of the sensors on the body are more easily made consistent between tests. In other embodiments, sensor(s) are attachable at any point along the length of the front extension 20 and/or rear extension 30.

As would be clear, the at least one connecting member 40A, 40B can have any suitable form. In FIG. 1, the connecting members 40A, 40B are shown as straps that connect around a subject's sides. Such straps would fit around the subject's torso, under their arms. However, in some embodiments, a harness or yoke-type structure may be used, rather than straps that fit under the arms. In such harness embodiments, at least one connecting member 40 extends over at least one of the subject's shoulders. Harness-type embodiments are discussed in more detail below. Some embodiments, moreover, include both harness-type structures and connecting members that fit around the torso under the arms.

In addition to straps, the at least one connecting member 40 can also comprise any form of buckle, fastener, and/or adjustors. As well, the at least one connecting member 40 may be flexible or rigid, depending on the embodiment. The at least one connecting member 40 can comprise any suitable attachment for holding the device in place on the subject during testing. In some embodiments where the connecting member is rigid, moreover, the connecting member 40, front extension 20, and rear extension 30 are formed together as a single rigid yoke (i.e., having a unitary construction).

It should also be clear that the front extension 20, rear extension 30, and at least one connecting member 40 are to be made of a non-electrically-conductive material, such as plastic, rubber, etc., to mitigate electrical interference with the measured response data.

In some embodiments, there is only one front sensor 50 on the front extension 20 and only one rear sensor 60 on the rear extension 30. In other embodiments, at least one of the front extension 20 and the rear extension 30 comprises multiple sensors. As well, in some embodiments, the front extension 20 and the rear extension 30 have equal numbers of sensors, while, in other embodiments, the front extension 20 and the rear extension 30 have different numbers of sensors.

The sensors 50 and 60 are, in some embodiments, a single type and/or model of sensor. In other embodiments, the front sensor(s) 50 are all one type and/or model of sensor while the rear sensor(s) 60 are all a different type and/or model of sensor. In still further embodiments, the front sensor(s) 50 may comprise multiple types and/or models of sensor, as may the rear sensor(s) 60. However, the sensors 50 and 60 are preferably suitable for dry application. That is, the sensors 50 and 60 are preferably suitable for conducting charges directly from dry skin without the use of intermediary gel. As well, in some embodiments, the sensors are individually detachable from the device 10, such that replacement sensors can be attached as/if desired.

The control unit 70 is electrically connected to all of the sensors on the device. The control unit 70 coordinates the delivery of power to the stimulating sensor(s) (e.g., to the front sensor(s) 50) and thus, in some embodiments, the control unit 70 comprises a battery unit. The battery unit, in some embodiments, is a rechargeable battery, while in other embodiments, the battery must be replaced once it has discharged. In still other embodiments of the device, the control unit 70 does not contain a battery. In such embodiments, power for the stimulus pulses is provided by an external power source through attached cables. Such embodiments are less portable than battery-enabled embodiments.

The control unit 70 also gathers the response data from the responding sensor(s) (e.g., from the rear sensor(s) 60) and communicates that data to a processor 80. In some embodiments, the control unit 70 preprocesses the data before sending to the processor 80. In other embodiments, further, the control unit 70 comprises an internal processor for analyzing the gathered data. In some such embodiments, the processor 80 merely functions as a display for results provided by the control unit 70. For example, in some embodiments, the processor 80 is built-in to the device 10 and comprises a display visible to the subject, such that no external computing devices are required for the subject to view the results of a scan. However, such embodiments require the device 10 to have higher processing power than embodiments in which neither the control unit 70 nor an internal processor 80 conducts analysis of the data.

Additionally, in some embodiments, the control unit 70 comprises multiple units. For example, in some embodiments, a front control unit delivers power to the front sensor(s) 50 while a rear unit gathers and/or analyzes data from the rear sensor(s) 60.

As should be clear, although a laptop is shown in FIG. 1, the ‘processor’ 80 can have any suitable form. For example, the processor 80 can comprise a desktop computer; a laptop computer, tablet, smartphone or other mobile computing device; a server or mainframe computer; and so on. In a preferable embodiment, the processor 80 is able to display results directly to the subject of the device 10. However, in other embodiments, the processor 80 analyzes the data and passes the results to a third party (e.g., a health care provider or wellness coach) or simply stores the results of the analysis for later review.

In some embodiments, the processor 80 communicates with the control unit 70 over a wireless connection. The control unit 70 and the processor 80 may communicate using any suitable protocol or channel, including WiFi channels, Bluetooth, etc. In other embodiments, the control unit 70 communicates with the processor 80 over a wired connection.

In some embodiments, as mentioned above, the sensors are activated in a predetermined sequence. In one such embodiment, where the front extension 20 and the rear extension 30 each comprise multiple sensors, a first stimulus pulse is delivered through the uppermost front sensor 50 on the front extension 20. Responses to that first stimulus pulse are gathered from each of the rear sensors 60. A stimulus pulse is then delivered to the subject through the next-highest front sensor 50 on the front extension 20, and the response of each rear sensor to that stimulus pulse is gathered. This is repeated for each of the front sensors. As a non-limiting example, where there are seven front sensors 50 and seven rear sensors 60, forty-nine responses would be gathered during the activation sequence. Where there are seven front sensors 50 and only one rear sensor 60, seven responses would be gathered. However, as should be clear, any suitable stimulation sequence may be applied.

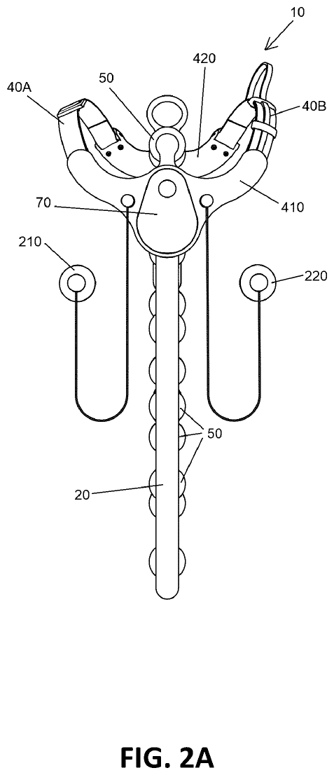

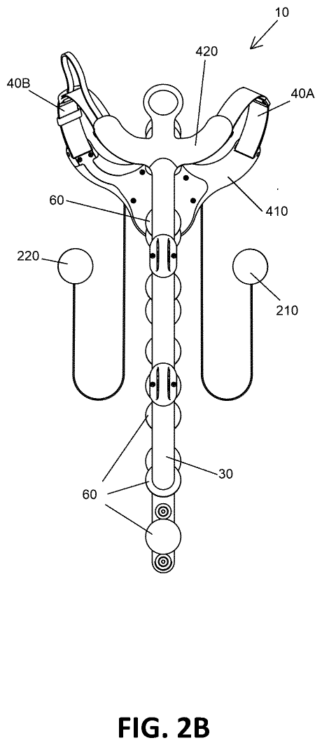

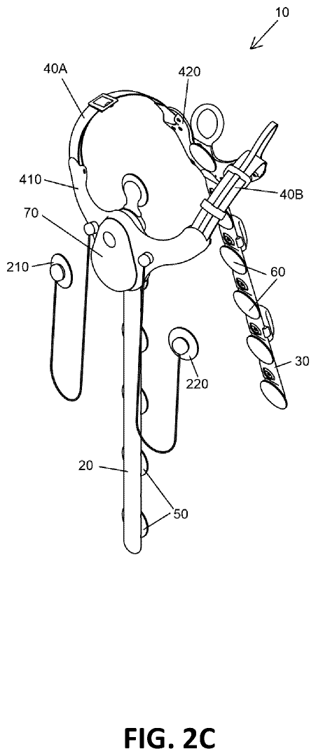

In one embodiment of the harness-type device 10, shown in FIGS. 2A to 2C, a chest piece 410 and a back piece 420 are held together by connecting members 40A, 40B and fit over the subject's head. As can be seen, in this embodiment, the connecting members 40A, 40B comprise adjustable buckles and straps. (Note that, in some embodiments, the chest piece 410 and back piece 420 are directly attached together, e.g., as a collar around the subject's neck, without additional connecting members 40. As well, some embodiments of the invention have a chest piece but no back piece, or a back piece but no chest piece. In an embodiment with only a chest piece 410, the front extension 20 extends from the chest piece 410 and the chest piece 410 is connected directly to the rear extension 30 by the at least one connecting member 40. Similarly, in an embodiment with only a back piece 420, the rear extension 20 extends from the back piece 420 and the back piece 420 is connected directly to the front extension 30 by the at least one connecting member 40.) This embodiment also comprises additional sensors 210 and 220. As can be seen, these additional sensors 210, 220 are not attached to the front extension 20 or back extension 30. Rather, the additional sensors 210, 220 are electrically connected to the control unit 70 by the cables shown. The additional sensors can be positioned at a subject's forehead and back of the neck, at the subject's hands and/or feet, and so on. Further, although only two additional sensors are shown in this embodiment, it should be clear that the device may comprise any desirable number of such additional sensors, to be positioned at any desirable position.

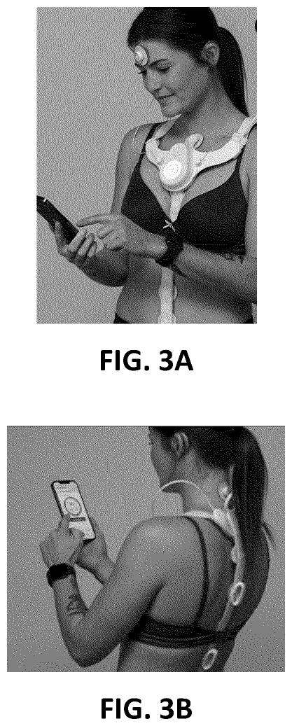

FIGS. 3A and 3B show the device 10 of FIGS. 2A to 2C in use by a subject. As can be seen, the front extension 20 generally bisects the front of the subject's torso and the rear extension 30 generally follows the line of the subject's spine. A forehead sensor (i.e., an additional sensor 210) is in position on the subject's forehead (shown in FIG. 3A) and a neck sensor (i.e., an additional sensor 220) is in position on the back of the subject's neck (shown in FIG. 3B).

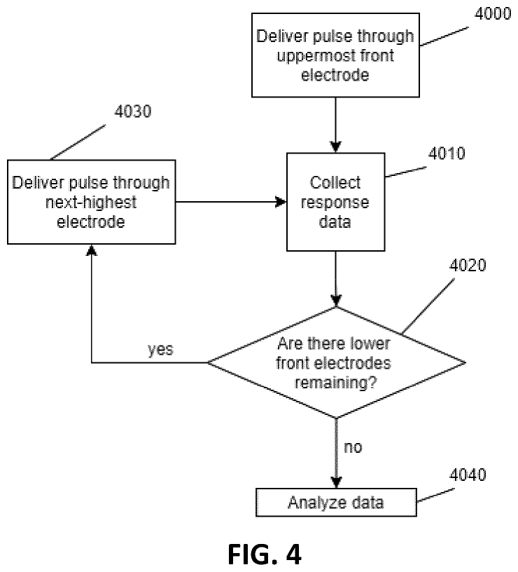

FIG. 4 is a flowchart detailing a method according to an aspect of the invention. At step 4000, a pulse is delivered through the uppermost front sensor. An electrophysiological response of each rear sensor(s) to that pulse is gathered at step 4010. If there are still front sensors remaining at step 4020 (i.e., front sensors through which a pulse has not yet been delivered), a pulse is delivered through the next-highest front sensor at step 4030. The response(s) of the rear sensor(s) are then again gathered at step 4010. Steps 4020, 4030, and 4010 are repeated as long as there are front sensors that have not yet delivered a pulse. Once each front sensor has been used to deliver a pulse, the collected data is analyzed to determine a response at step 4040. It should also be noted that, in some embodiments of the method, the analysis of data at step 4004 is performed in parallel with the stimulation and collection of new data at steps 4010 to 4030. Again, however, it should be noted that the use of the uppermost front sensor to deliver the first pulse is merely one embodiment, and that other activation sequences may be used in other embodiments.

As noted above, for a better understanding of the present invention, the following references may be consulted. Each of these references is hereby incorporated by reference in its entirety:
[1] Berthoud H R, Neuhuber W L. Functional and chemical anatomy of the afferent vagal system. Auton Neurosci. 2000 Dec. 20;85(1-3):1-17.
[2] Szopinski J, Pantanowitz D, Jaros G G. Diagnostic accuracy of organ sensorrmal diagnostics. A pilot study. S Afr Med J. 1998 February;88(2):146-50.
[3] Szopinski J Z, Pantanowitz D, Lochner G P. Estimation of the diagnostic accuracy of organ sensorrmal diagnostics. S Afr Med J. 2004 July;94(7):547-51.

As used herein, the expression “at least one of [x] and [y]” means and should be construed as meaning “[x], [y], or both [x] and [y]”.

It should be clear that the various aspects of the present invention may be implemented in connection with software modules, such as software for processing the gathered response data from the sensor(s) and/or software for controlling the delivery of stimulus pulses. As such, the present invention may thus take the form of computer executable instructions that, when executed, implement various software modules with predefined functions.

Software related to embodiments of the invention may be executed by a computer processor or similar device programmed in the manner of method steps, or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Similarly, software related to embodiments of the invention may be implemented in any conventional computer programming language. For example, preferred embodiments may be implemented in a procedural programming language (e.g., “C” or “Go”) or an object-oriented language (e.g., “C++”, “java”, “PHP”, “PYTHON” or “C #”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A device for measuring an electrophysiological response of a subject to predetermined electrical stimulus, said device comprising:
a front extension;
a rear extension;
at least one connecting member, said at least one connecting member connecting said front extension and said rear extension;
at least one front sensor attached to said front extension; and
at least one rear sensor attached to said rear extension,
wherein, when said device is in use:
said front extension extends vertically down a front of said subject's torso;
said rear extension extends vertically down said subject's back; and
said at least one front sensor and said at least one rear sensor are in electrical contact with said subject's skin,
and wherein a processor coupled with the device determines said electrophysiological response based on data from said at least one front sensor and said at least one rear sensor.

2. The device according to claim 1, further comprising a control unit that:
directs the delivery of power to at least one of said at least one front sensor and said at least one rear sensor; and
gathers data from at least one of said at least one front sensor and said at least one rear sensor.

3. The device according to claim 1, wherein a number of sensors on said front extension and a number of sensors on said rear extension are equal.

4. The device according to claim 1, wherein said at least one front sensor and said at least one rear sensor each comprises a plurality of sensors.

5. The device according to claim 1, wherein said at least one front sensor and said at least one rear sensor are positioned at predetermined locations on said subject's torso.

6. The device according to claim 1, wherein said at least one connecting member comprises a strap.

7. The device according to claim 1, wherein at least one of said front extension and said rear extension is conformable to said subject's body.

8. The device according to claim 1, wherein said device further comprises a chest piece, said front extension extending from said chest piece, and said chest piece being connected to said rear extension by said at least one connecting member.

9. The device according to claim 1, wherein said device further comprises a back piece, said rear extension extending from said back piece, and said back piece being connected to said front extension by said at least one connecting member.

10. The device according to claim 1, wherein said device further comprises a harness, said harness having a chest piece and a back piece, said chest piece and said back piece being connected by said at least one connecting member, said front extension extending from said chest piece and said rear extension extending from said back piece, and said harness being positioned over at least one shoulder of said subject when said device is in use.

11. The device according to claim 10, wherein said harness has a unitary construction.

12. The device according to claim 4, wherein said plurality of sensors are activated in a predetermined activation sequence.

13. The device according to claim 12, wherein said sequence comprises:
an activation of a sensor on said front extension to thereby provide a first stimulus pulse, and
a determination of a first response, said first response being a response to said first stimulus pulse, from each said at least one rear sensor,
wherein activations and response-determinations are performed for each other sensor on said front extension such that responses from each said at least one rear sensor are determined for each at least one front sensor.

14. The device according to claim 12, wherein said sequence comprises:
an activation of a sensor on said rear extension to thereby provide a first stimulus pulse, and
a determination of a first response, said first response being a response to said first stimulus pulse, from each said at least one front sensor,
wherein activations and response-determinations are performed for each other sensor on said rear extension such that responses from each said at least one front sensor are determined for each at least one rear sensor.

15. The device according to claim 1, further comprising at least one additional sensor, wherein said at least one additional sensor is connected to a control unit on said device and wherein said control unit is in communication with said processor.

16. A method for measuring an electrophysiological response of a subject to predetermined electrical stimulus, said method comprising:
activating a sensor positioned at a torso of said subject to thereby provide a first stimulus pulse; and
determining a response to said first stimulus pulse from at least one sensor positioned at a back of said subject.

17. The method of claim 16, wherein a plurality of front sensors are positioned at said subject's torso and wherein steps (a) and (b) are separately repeated for each of said plurality of front sensors.

18. The method of claim 17, wherein each of said plurality of front sensors is vertically aligned with other sensors of said plurality of front sensors, and wherein said plurality of front sensors extends down said subject's torso.

19. The method of claim 18, wherein a uppermost sensor of said plurality of front sensors is activated first and each next-highest sensor of said plurality of front sensors is activated in sequence.

20. The method of claim 17, wherein a number of said at least one sensor positioned at said subject's back is equal to a number of said plurality of front sensors.

21. A method for measuring an electrophysiological response of a subject to predetermined electrical stimulus, said method comprising:
activating a sensor positioned at a back of said subject to thereby provide a first stimulus pulse; and
determining a response to said first stimulus pulse from at least one sensor positioned at a torso of said subject.
